(19) United States
(12) Patent Application Publication
(10) Pub. No.: US 2004/0064974 A1
(43) Pub. Date: Apr. 8, 2004

(54) Mechanical support which can be arched, distorted, rotated and deformed

(76) Inventor: Wilhelm Schuster, Linz (AT)

Correspondence Address: Grant D Kang, Husch & Eppenberger, 190 Carondelet Plaza, St Louis, MO, 63105, US

(21) Appl. No.: 10/451,122

(22) PCT Filed: Dec. 20, 2001
(86) PCT No.: PCT/EP01/15139

(30) Foreign Application Priority Data
Dec. 21, 2000 (AT) 2123/2000
Oct. 15, 2001 (AT) 1618/2001

(51) Int. Cl. A43B 7/22; A61F 5/14

(52) U.S. Cl. 36/91; 36/155

(57) ABSTRACT

The invention relates to a support especially for medical or technical purposes comprising at least one flexible element which can be arched and which comprises at least one central part and lateral elements, the arch of said element being adjustable by means of a device. According to the invention, even in its arched state, the support can be at least partially spatially distorted, and/or rotated, and/or deformed about an axis by means of at least one lateral element and/or central element.

The invention relates to a support, in particular for medical or technical applications, comprising at least one flexible element, which can be arched and which comprises at least one central element and lateral elements, the setting and/or arching of said element being adjustable by means of a device.

A support of this type is known from EP 0 485 483 B1.

From the literature a number of arching mechanisms are known which allow an element, which may be arched, in particular for lumbar support, to be adjusted to different degrees of arching. In addition, level adjustment of the arching apex may also be provided. Apart from the adjustment of arching and, as the case may be, the adjustment of the level of the arching apex, no further settings or adjustments are possible.

It is the object of the invention to so design a support according to the generic clause of claim 1 that in addition further space-related actions are possible, in particular also after the arching has been set. A further object resides in expanding the field of application of such supports, in particular also with regard to the manufacture of shoes comprising loose or permanently integrated adjustable supports.

This object is attained according to the invention in that, even in its arched state, at least one lateral element and/or the central element and/or the arched region may be at least partly spatially distorted and/or rotated and/or deformed about an axis.

This should have the effect that a support serving, for example, as a transverse arching support, a bodice, a permanently integrated or loose shoe arch-support, prosthesis, orthesis, implant, casing, machine element or element for apparatus engineering or the like, prior to arching as well as during and after setting the degree of arching by means of at least one lateral element, may be partly spatially distorted and/or rotated about one or more axes placed at angles to one another and/or may be able to change within a straight plane or spatially bent, in particular spatially, even in an upright position.

Further advantageous embodiments of the invention are apparent from the subsidiary claims.

In what follows the invention is elucidated in detail by way of working examples.

There is shown in:

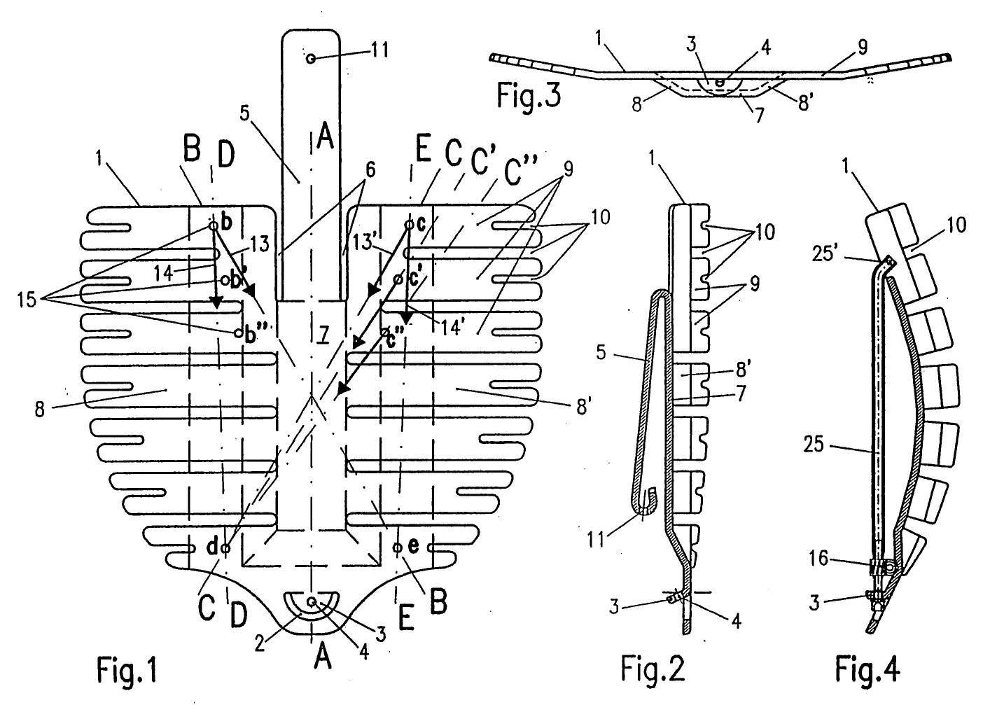

FIGS. 1 to 3 various illustrations of a first embodiment of a support,

FIG. 4 a second embodiment of an arched support,

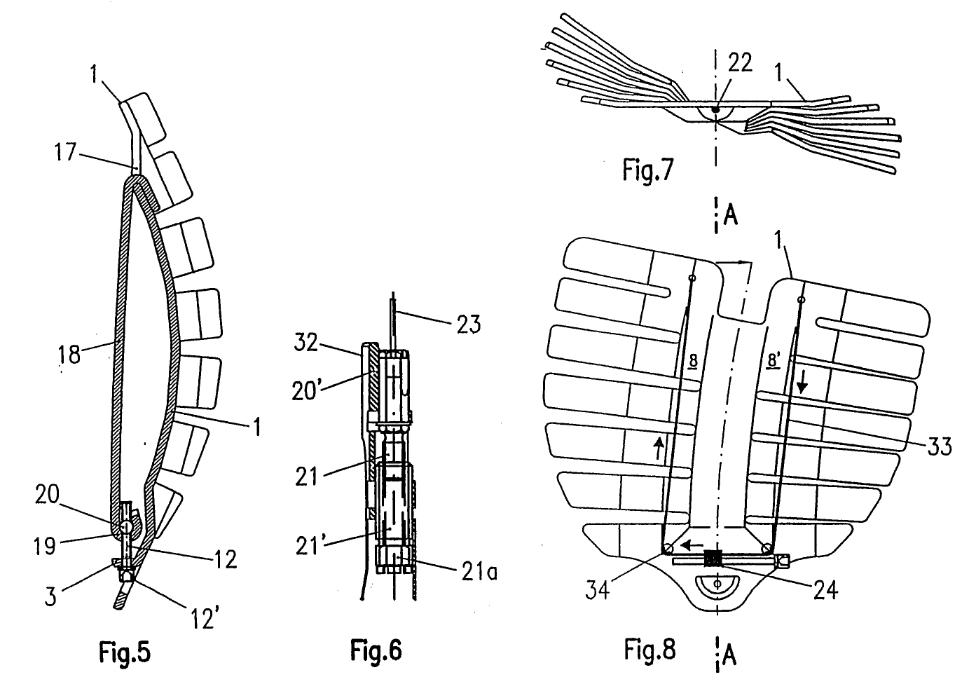

FIG. 5 a third embodiment of an arched support,

FIG. 6 a double-threaded tensioning element

FIG. 7 a forth embodiment of a support distorted about its own longitudinal axis,

FIG. 8 an embodiment of a fifth support deformed in its upright position,

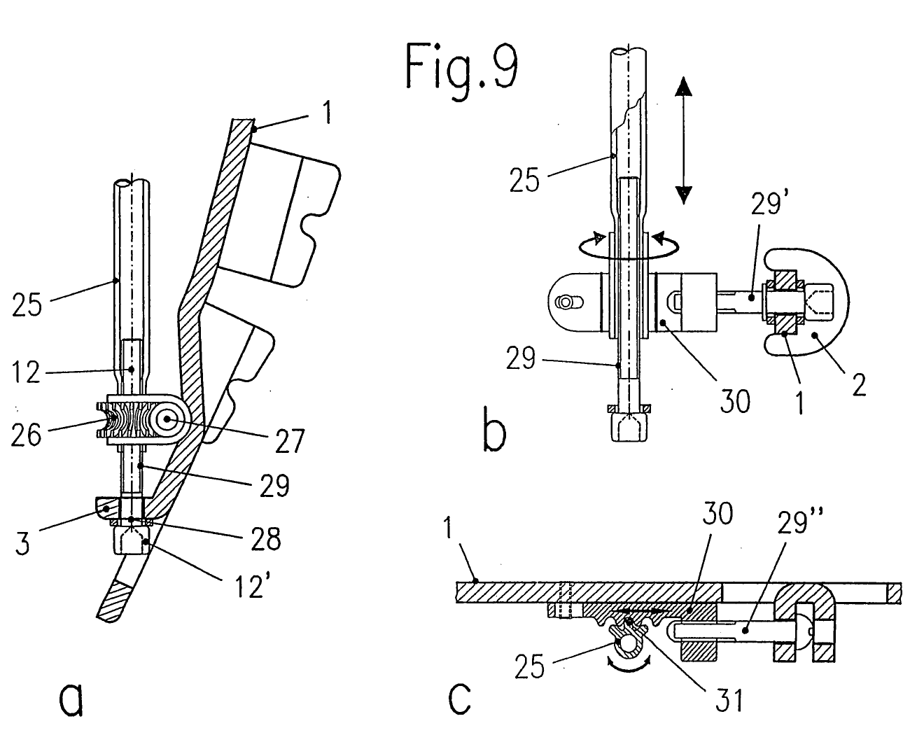

FIG. 9 embodiments of tensioning, pressure, rotation, distortion elements,

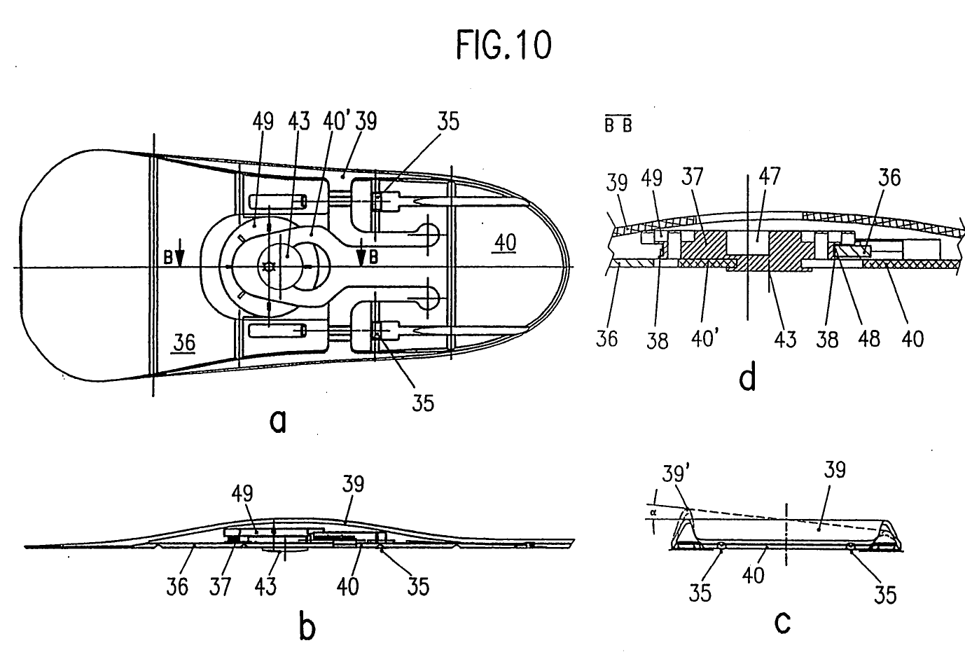

FIG. 10 an embodiment of a shoe insert which may be arched and distorted,

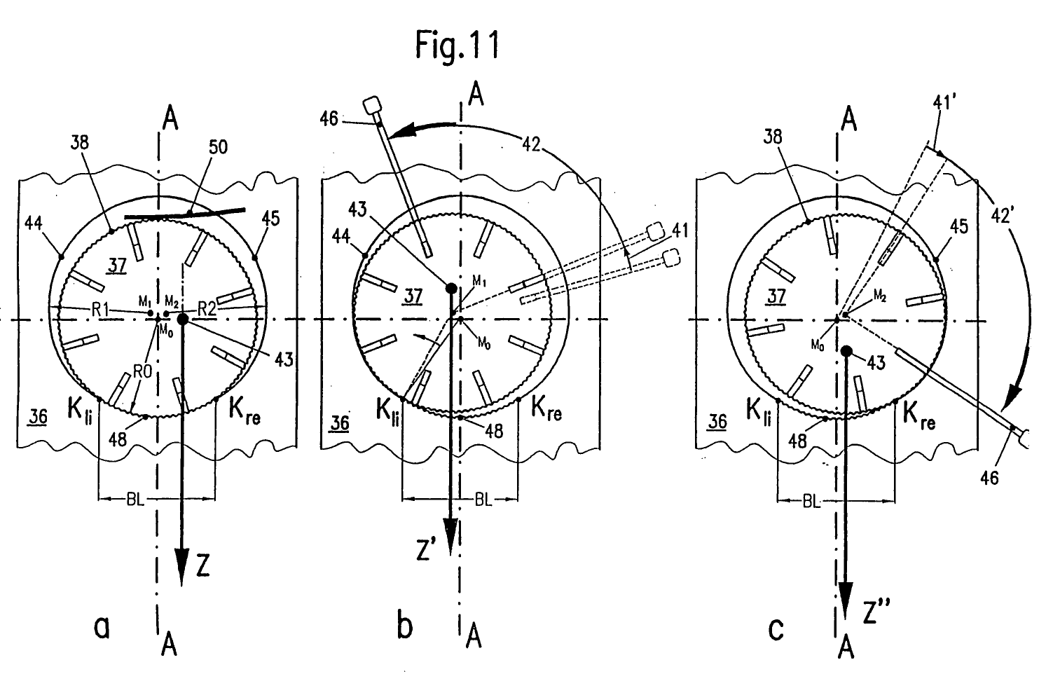

FIG. 11 an embodiment of a self-locking eccentric tensioning or adjusting mechanism,

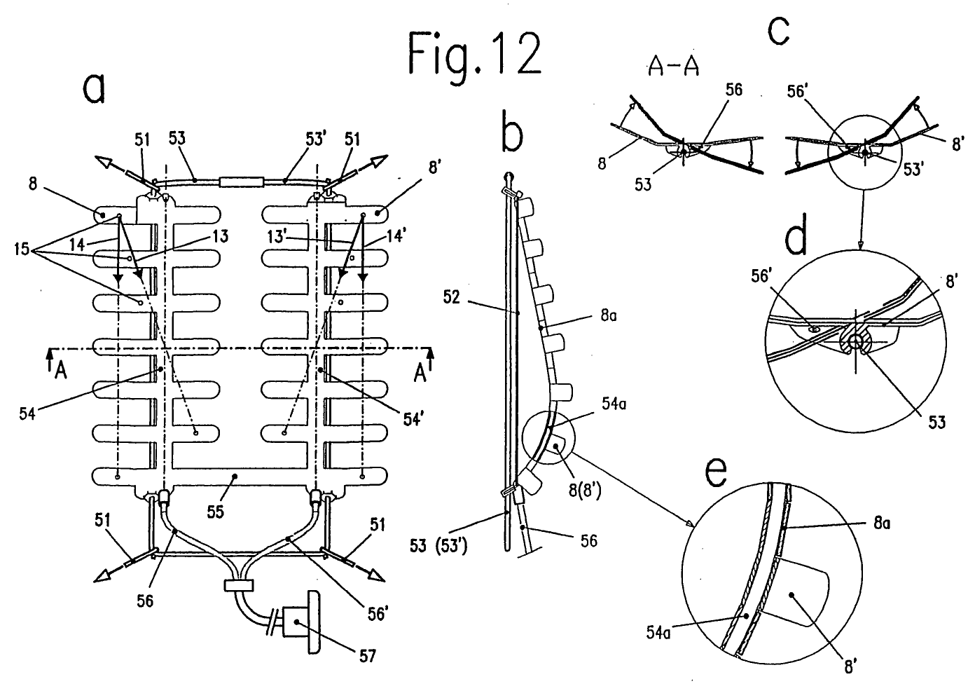

FIG. 12 a plurality of illustrations of an embodiment of a double arching-, distortion-rotation, support,

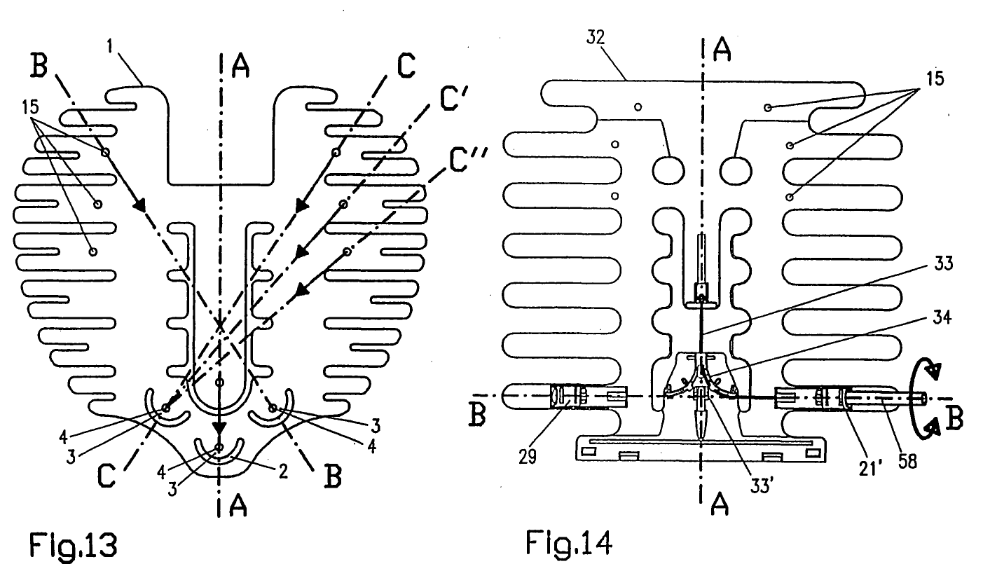

FIG. 13 an embodiment of a transverse arching or twisting support,

FIG. 14 an embodiment of an arching-twisting mechanism comprising optionally single support/deflectable traction, tensioning or adjusting elements,

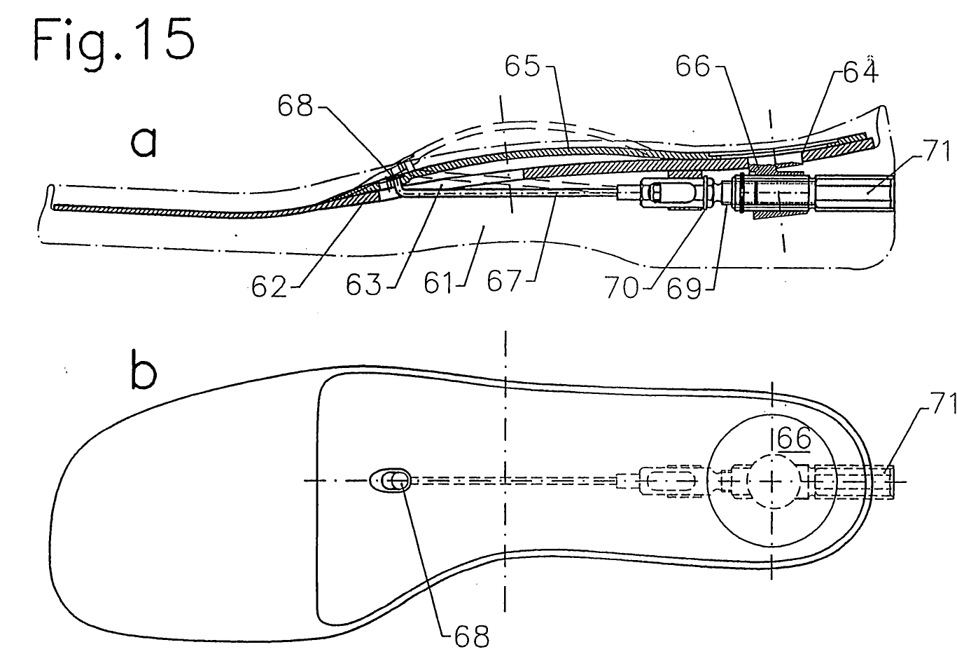

FIGS. 15a-b an integrated support in the sole portion of a shoe, a) in section through the central longitudinal axis and b) in plan view,

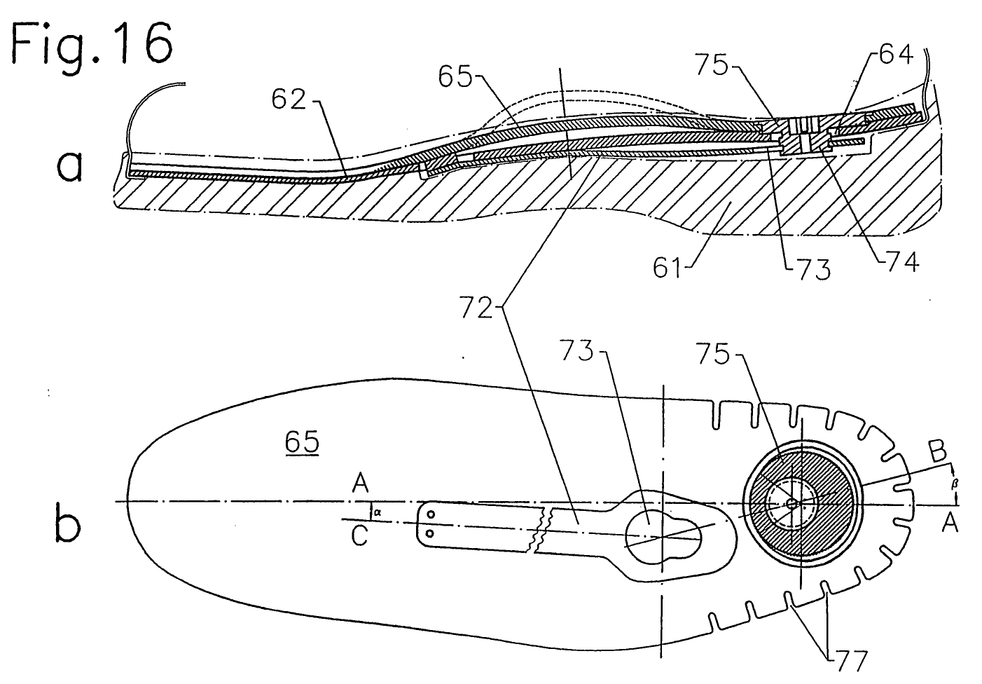

FIG. 16 a second embodiment of an integrated support in a shoe comprising an eccentric arrangement and a tongue to be hooked in, a) in section and b) in a view from below onto the arching strip,

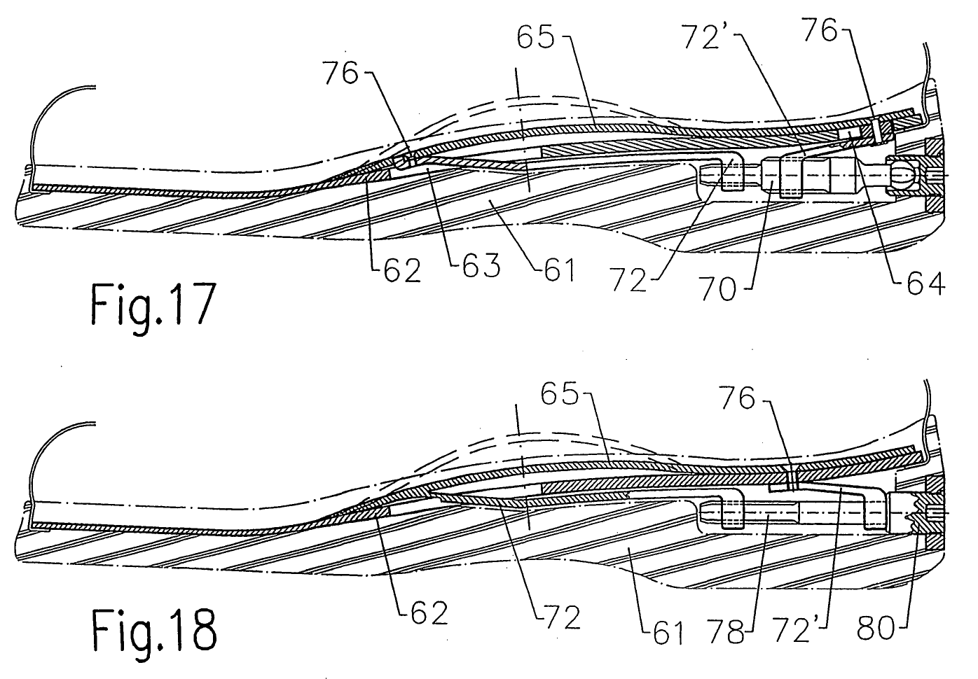

FIG. 17 a third embodiment of an integrated support in a shoe comprising an arching device in the heel region,

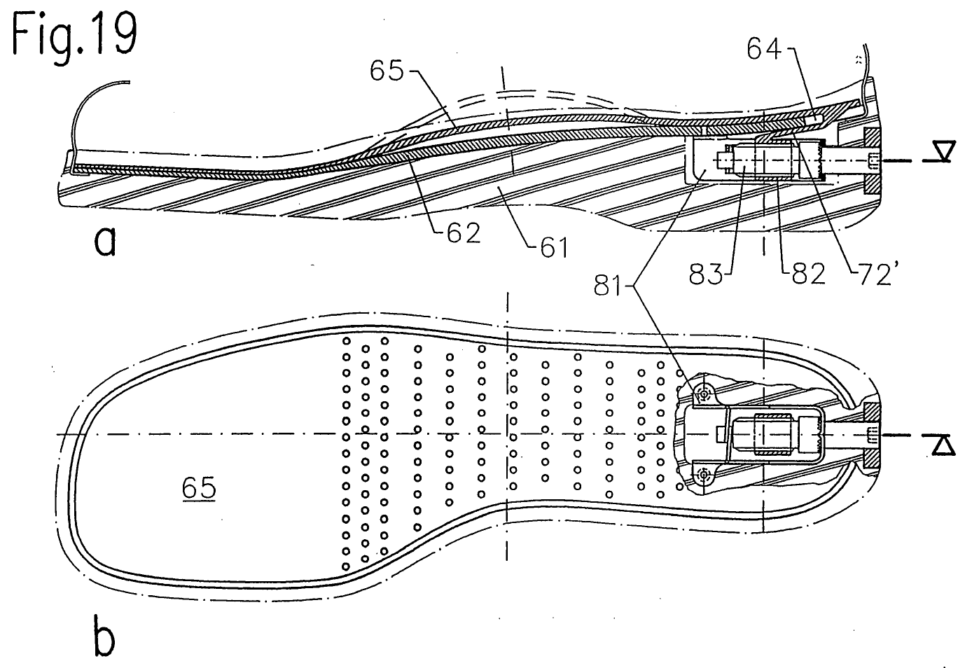

FIG. 18 a fourth embodiment of an integrated support in a shoe comprising a further arching device in the heel region,

FIGS. 19a,b a fifth embodiment of an integrated support in a shoe comprising a third arching device in the heel region, a) in section and b) in plan view,

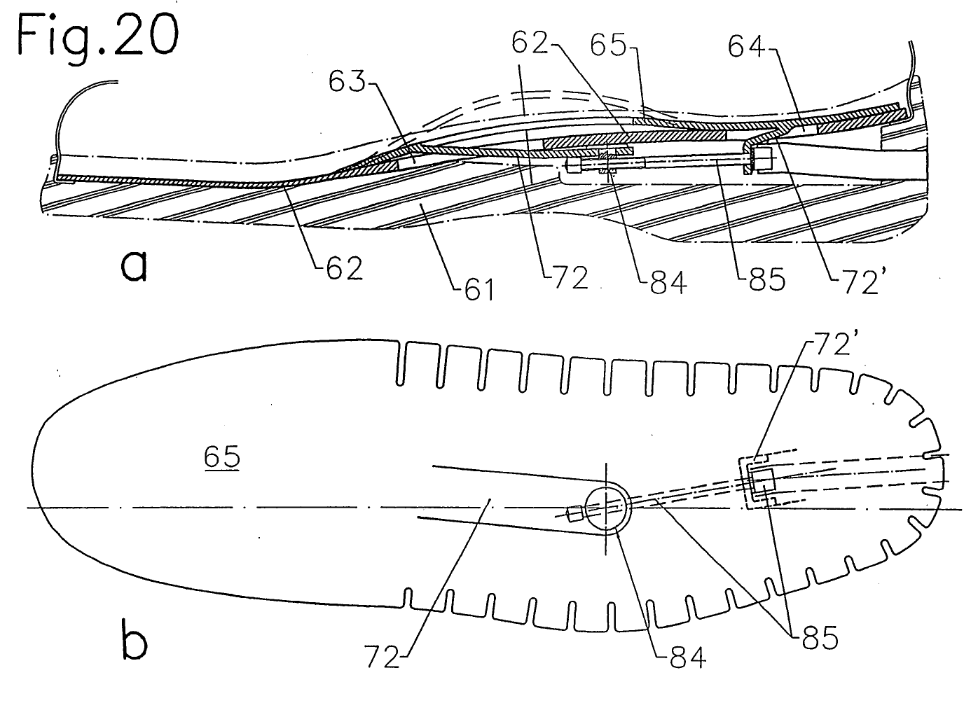

FIGS. 20a,b a sixth embodiment of an integrated support in a shoe, a) in section and b) in plan view,

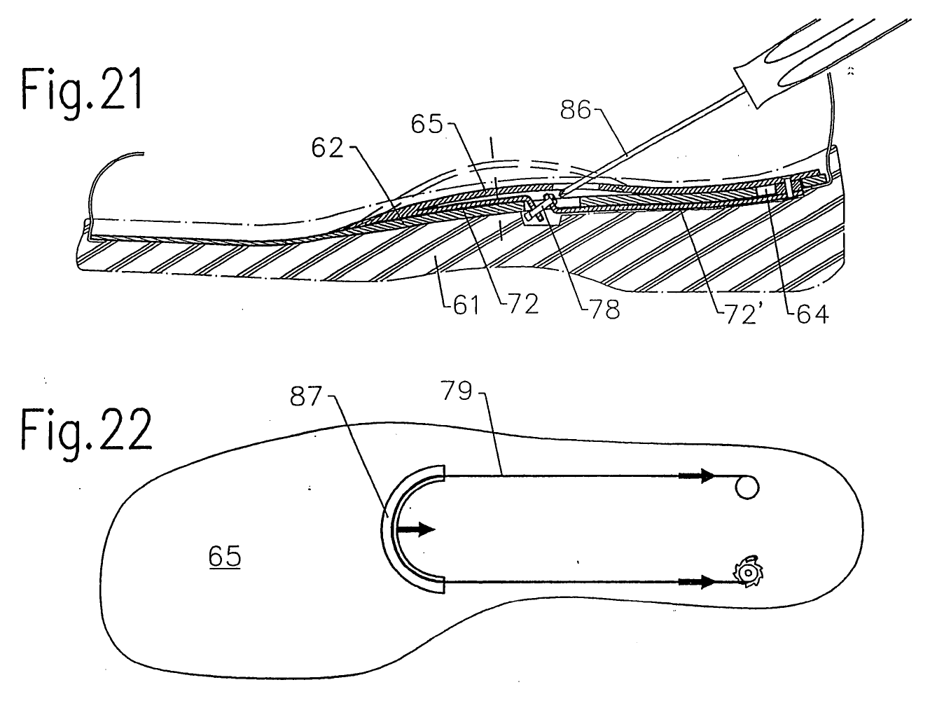

FIG. 21 a seventh embodiment of an integrated support in a shoe in section,

FIG. 22 an eighth embodiment of an integrated support in a shoe comprising an operating cable,

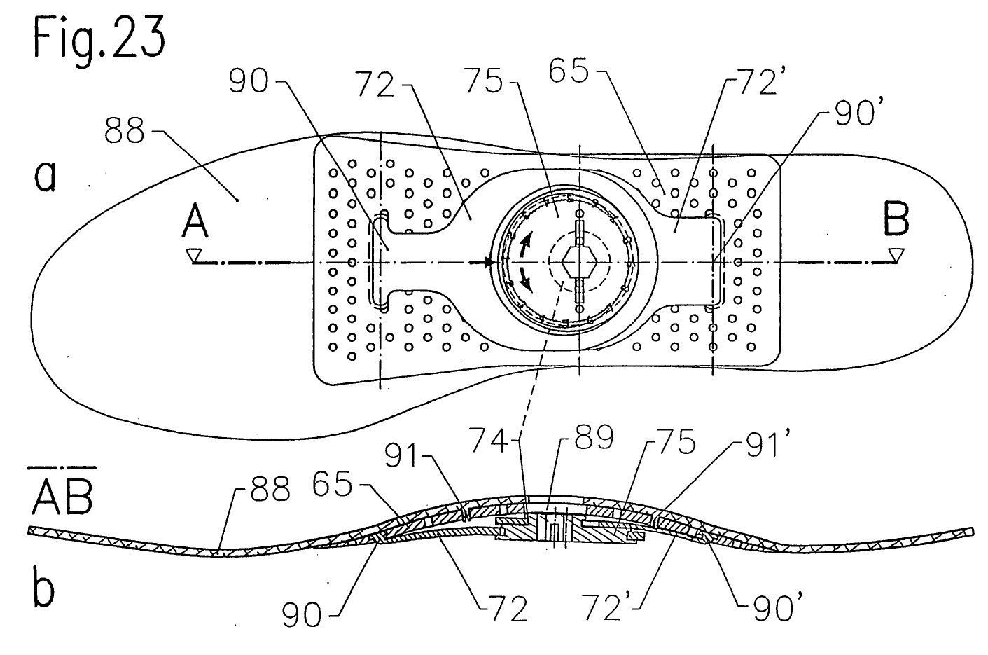

FIG. 23a an arching-twisting support for a foot arch on the underside of an integrated cover sole with perforated short arching-twisting strip and self-locking eccentric wheel-, traction-, arching adjustment with upper/lower drive mechanism,

b the same in longitudinal section side elevation (plane AB),

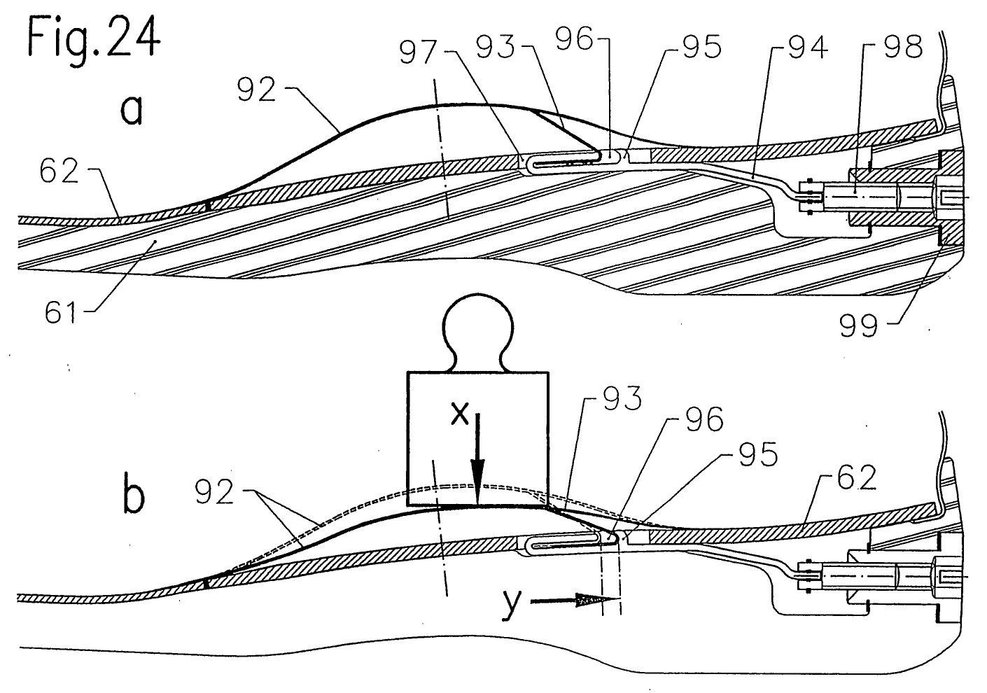

FIG. 24a a side elevation in longitudinal section through a shoe comprising a pre-arched spring arching strip, tractional de-arching screw and additional de-arching space,

b the same support according to FIG. 24, weighted, and with comfort free space utilisation,

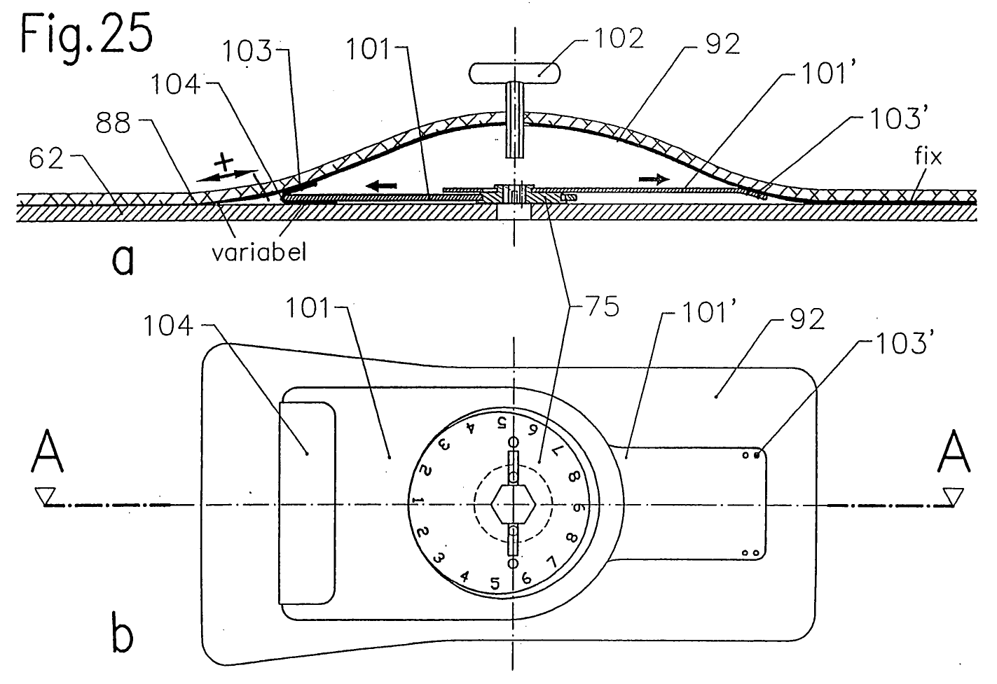

FIG. 25a a similar, pre-arched support in a longitudinal sectional side elevation with eccentric wheel-expansion (pressure) de-arching with comfort free space in the front,

b the de-arching distortion support according to FIG. 25 a viewed from below without insole and cover sole,

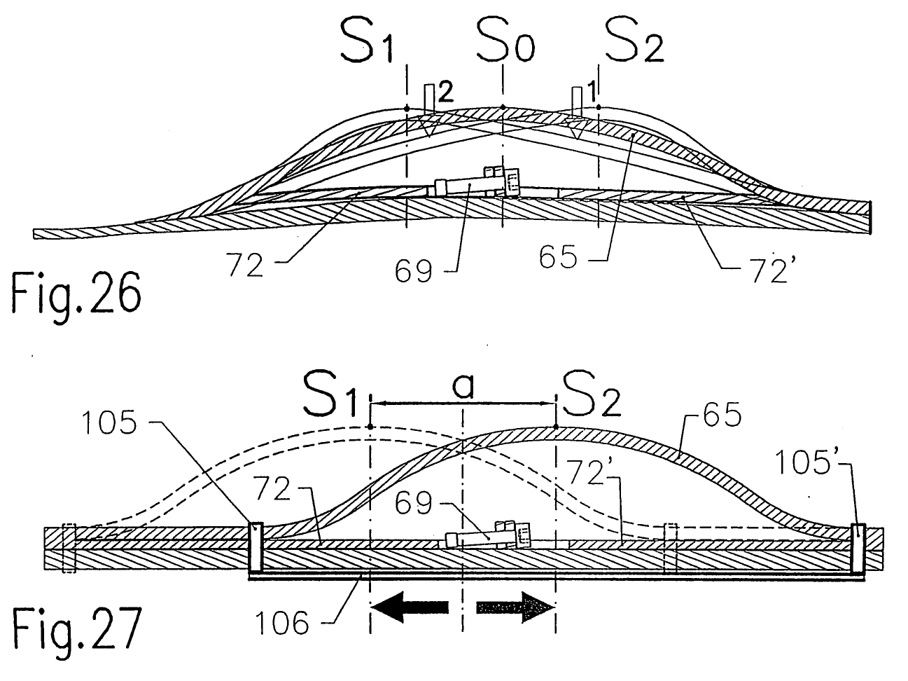

FIG. 26 a traction, arching, distortion support with automatic apex shifting,

FIG. 27 an arching, distorting support with mechanically-controllable apex shifting by way of sliding brackets with the level of arching remaining constant,

FIGS. 28a,b a longitudinal and cross-section through a traction, arching, distortion support comprising massage elements, a free space and additional integrated toothed wheel and worm gear arching adjustment,

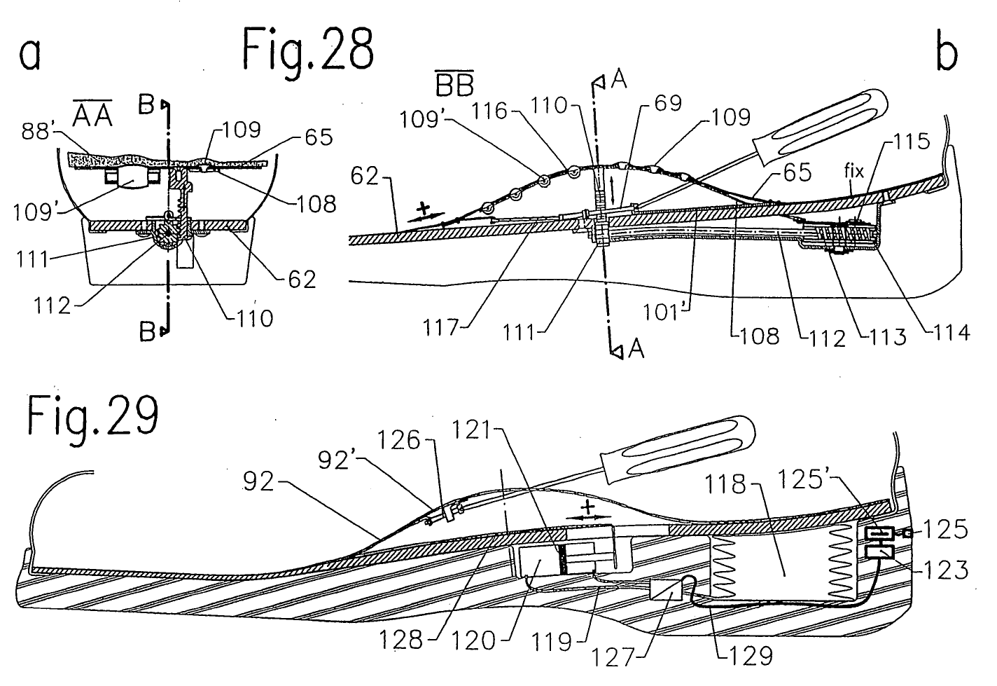

FIG. 29 a support with divided, overlapping arching strip halves and additional integrated compressed air automatic arching action,

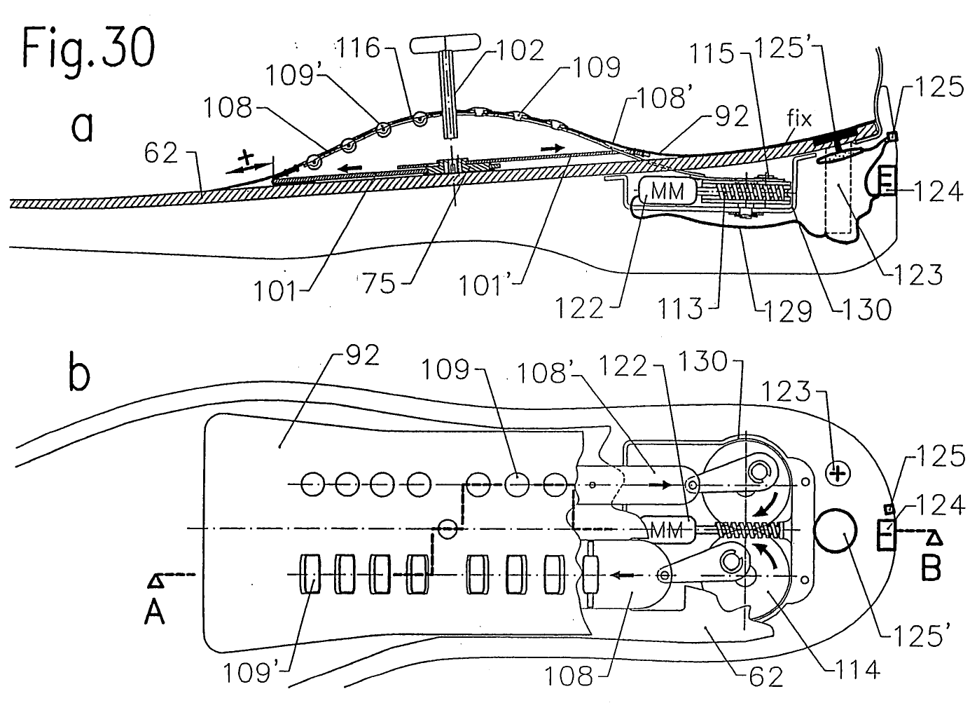

FIG. 30a a support with pre-arched spring arching strip, expansion elements, de-arching free space, massage elements and integrated motor with internal/external energy source together with automatic chip control, built into a shoe, shown in section,

b the same as in FIG. 30a in plan view with a stepwise view through the massage arching strip and the insole onto the integrated worm gear motor automation.

FIGS. 1 to 3 show an embodiment of the invention, manufactured for a support element from a single piece symmetrical strip 1, consisting of a resilient material, e.g. sheet metal, synthetic material, manufactured by blanking, milling or injection moulding. The strip 1 comprises a deflectable lower holding element 3 limited by a semicircle 2 comprising an aperture 4 as well as brackets b, b′, b″, c, c′, c″ d and e designed e.g. in the form of pins or even holes for fixing tensioning, adjusting or holding devices.

At the upper end of the strip 1 in its central axis A-A an extension 5 is provided, which is separated on both sides from the upper web 9 of the side wings 8, 8′ by slits 6. Starting from the central part 7, the strip 1 consists in this case of two symmetrical side wings 8, 8′, consisting of one or a plurality of webs 9, which may be subdivided by at least one shorter or longer transverse slit 10, extending transversely to the central part 1, in which context the webs 9 of the side wings 8, 8′ in the embodiment (FIG. 1) are essentially of a length decreasing from the top towards the bottom.

Along the broken connecting line between the lowest points of the slits 6 limiting the extension 5, the latter is bent, in the present case backwards, as is apparent from FIG. 2, in which case its end section, bent once again in the present case, is provided with a second holding element for a tensioning or holding device, designed in the present case again as an aperture 11 (see FIG. 2). Between the two apertures 4 and 11 a traction, pressure, distortion, tensioning, adjusting or fixing means (e.g. according to FIGS. 4, 5, 6, 8 and 9)—not shown in the present example—may be hooked in, making it possible to change the shape of the strip 1 (e.g. an arching, see FIG. 4).

Due to the configuration of the side wings 8, 8′ (FIG. 1), which may be further subdivided by additional transverse slits 10, they yield passively under load, e.g. under pressure. By pressure and/or tensioning elements connecting them—illustrated symbolically in the present case only by arrows 13, 13′, 14, 14′—they are, depending on the type of connection and setting, wholly or partially spatially deformable, e.g. deflectable, individually or in groups, in which context tensile or compressive-force arrangements are possible, e.g. of b, b′ or b″ diagonally e.g. in the axis BB in relation to e or of c, c′, c″ diagonally e.g. in the axis CC in relation to d or of point c in the axis EE to point e or of b in the axis DD. The tensile or compressive forces may engage symmetrically or asymmetrically only on one wing 8 or 8′ or on both wings 8 and 8′.

For this purpose the strip 1 also comprises, for example, a number of fixing points 15 (see FIG. 1), where one or a plurality of stationary or lengthwise adjustable traction or pressure elements 13 and 13′, for example, for spatial distortion or rotation and 14, 14′ for deformation may be arranged in a stationary or detachable manner, preferably so that they may be hooked in, so that at different angles spatial distortions, rotations or deformations may be performed and may thereafter, if the need arises, be changed as desired (see, e.g. the arrows in FIG. 1).

FIG. 3 shows a view from below onto the strip 1, from which the protruding lower holding element 3 with its aperture 4 can be seen. Preferably in the central region of the strip 1 there are provided in the central element 7 deflections of the side wings 8 and 8′, directed towards the interior (rear), for example in the form of a recess, groove or trough. By such, e.g. central deflections or bends, for example, direct abutting of spinous processes of a spine may be avoided. In FIG. 1 broken lines represent examples of such deflections.

FIG. 4 shows a cross-section of a further support, which, apart from an arching adjustment, also shows a spatial distortion or twisting by means of a twisting mechanism 16 shown in FIG. 9. In this case the upper end of a rod 25 of the twisting mechanism 16 is made angular. The rod 29 is hooked in an articulated manner into the upper portion of the strip 1 by means of a retaining bolt 25′. Each rotational movement in the sense of a torsion or distortion transmitted to the distortion mechanism 16 is therefore transmitted to the strip 1.

FIG. 5 shows strip 1 in an arched state, where the traction element 18 with a hook-like anchor is loosely hooked into or fixed in an upper slit 17 and which at the lower end is connected to the holding element 3 by means of simple tensioning means 12, in which context the degree of convexity may be set by lengthening or shortening the tensioning means 12. In the embodiment an element 19 is bent in U-shape at the other (lower) end of the traction element 18, serving to accommodate a bracket 20. In the present case, the bracket 20 comprises an internal thread, into which a screw having an external thread may be screwed to serve as a tensioning means 12, the said screw being guided through the aperture 4 in the holding element 3. By screwing or unscrewing of the screw an adjustment of the arch is performed. Instead of an additional bracket 20 the lower portion of the traction element 18 itself may e.g. be bent or thickened and may be provided with a thread, into which/from which a screw serving as a tensioning means 12 may be screwed or unscrewed. With the rotatable—e.g. secured—positioning of the head of the screw serving as the tensioning means 12 in or on the holding element 3, screwing or unscrewing of the screw jointly with the traction element 18 brings about an adjustment of the convexity.

FIG. 6 illustrates a double-threaded tensioning element, wherein a longitudinally displaceable tensioning screw 21 with e.g. a hexagonal sliding shaft to prevent rotation, is rigidly connected in an irrotational manner to the arching strip 32, e.g. in a hexagonal sliding passage 20′. This tensioning screw 21 comprises in the present example a right-hand thread and engages with the latter in the internal right-hand thread of the double threaded screw 21′, comprising a left-hand thread on the outside, which engages in a left-hand thread passage, which is connected likewise as one piece or rigidly to the same arching strip 32. The hexagonal sliding shaft of the tensioning screw 21 may also serve for the releasable or permanent accommodation of the end section of a tensioning element, e.g. of a cable 23. By turning the double-threaded screw 21′ with its left-handed external thread in its stationary left-hand passage, which, rigidly connected to its base, is standing still, this screw 21 when turned to the right is pulled out of the passage instead of into the passage while at the same time the tensioning screw 21, which in its turn slides axially, but without distortion, is pulled automatically in the same direction of motion as the screw 21′. This means that when turned the traction or tensioning element, e.g. a cable 23, is tensioned or tension-released twice in the same longitudinal direction. As a result of the simultaneous interaction and action alongside one another of the two threads doubling of the adjustment path is attained per revolution with the normal thread pitch unchanged, without having to dispense with the self-locking action. The normal self-locking action of threads is not lost despite doubling the displacement distance per revolution.

The advantage of doubling the displacement distance per revolution with the self-locking action remaining constant is ensured even if the hexagonal sliding part of the screw 21 comprises a right-hand internal thread and the screw 21′ with its left-hand external thread instead of a right-hand internal thread has an additional pin with an identical right-hand external thread, which now engages the internal thread of the hexagonal sliding part.

FIG. 7 shows in plan view a simple manner of a twisting or rotation possibility of a strip 1, in which case its central axis 22 (see also A-A in FIG. 1) is shown unbent for reasons of simplicity. The rotation or twisting is likewise shown evenly, while uneven settings are also possible.

FIG. 8 shows an upright deformation of a strip 1, wherein, for example, by means of a deflection-traction element in the form of a cable 33, arranged in both wings 8, 8′ parallel or in any other way, e.g. obliquely in relation to one another, fitted in each case at the upper end, and an adjustment device 23, arranged in the lower portion—in the present case, centrally—, where necessary via deflection points 34, an upright deformation of the strip 1 is brought about in that at least one side of the deflection traction element, the cable 33, is shortened. This causes one of the two wings 8 or 8′ to be contracted so that the upright deformation shown in FIG. 8 in relation to the central axis A-A—regardless of an arching—may be performed. Instead of a central adjustment device 24, each side wing 8, 8′ may be adapted to be expanded or contracted, alternately as the case may be, by means of only one or even a plurality of pressure and/or traction elements, optionally attacking at different points.

FIGS. 9a, b, c are examples of adjustment devices, by means of which both a tractional or arching adjustment as well as a twisting or torsion or rotation and/or upright deformation of the support or of parts of the latter or of other bodies may be performed.

The two-fold adjustment device shown in FIG. 9a comprises a rod 25—hollow at least in its lower portion—, whereon a worm gear 26 is rigidly arranged, which combs with a worm 27. By turning the worm 27 a rotation of the worm wheel 26 takes place and, as a result, also of the rod 25. Due to an interaction of the upper end of the rod 25, which, e.g. in an angular manner and with a retaining bolt 25′, is in engagement with a strip 1 (see FIG. 4), a distortion or torsion of the upper portion of the strip 1 takes place as well when the rod 25 is rotated or likewise a rotation of individual transverse elements. In the lower, e.g. tapered end of the rod 25, which may also transmit longitudinal, tensile or compressive forces for adjusting the arching, one end of the tensioning means 12 is positioned in a rotatable manner or with a thread. The other end of the tensioning means 25 emerging from the rod 25, which may, for example, be a tensioning screw 29 or a Bowden or other tensioning or pressure adjusting mechanism, not shown, is passed through an abutment 28, e.g. the deflectable holding element 3 and is provided with a head 12′ of a tensioning screw 29 supported there against. By screwing or unscrewing the tensioning screw 29, for example also into/out of the lower end of the rod 25, a tensioning or arching adjustment is performed.

FIGS. 9b and c shows a different embodiment of the adjustment device. With regard to the adjustment of the convexity, it is designed analogously to FIG. 9a, whereas the rotational movement for twisting or torsion is brought about by a displaceable toothed rod 30, which is in engagement with a pinion 31 rigidly arranged on the rod 25. As a result of the stationary arrangement of the toothed rod 30, movable only in the transverse direction, the pinion 31, for adjusting the convexity, must be slidably arranged in the grooves of the toothed rod 30 or separate adjustment of the convexity may also be provided.

FIG. 10 shows a shoe insert serving as a support with a support strip 39 which may be arched and/or distorted, supported by a front section 36 and a rear section 40. Both sections 36 and 40 are provided with two adjusting screws 35, arranged e.g. parallel and in spaced apart relationship to one another and/or with an eccentric adjustment device, consisting of an eccentric device 43 and a tongue 40′ being in co-active engagement with the latter. This eccentric means is permanently or releasably fitted to a brake disk 37, which is connected to the latter by a likewise permanently connected adjusting ring 49. This brake disk in its turn is positioned in the section 36 of the casing and base strip, which on the front side of the shoe insert is connected to the cover or arching strip 39. The integrally formed base strip, the section 40, connected to the heel side, forms an integral part of it or is releasably in engagement with the tongue 40′ whose recess, closed on all sides, is in co-active engagement with the front section 36 of the casing or base strip by way of the eccentric means 43 and its brake disk 37. By turning the circular brake disk 37 in section 36 of the casing strip, the tongue 40′ is pulled or, conversely, released via the eccentric means 43 in such a manner that the support/arching/twisting strip 39 of the shoe insert is arched or de-arched or, respectively, the level of convexity set and fixed by means of its releasable self-locking action. This fixation of the respective setting is attained according to FIG. 11 a in that, after the adjusting force has been released, the brake disk 37 with its e.g. sinusoidal or otherwise formed rounded off friction surface 38 is pulled automatically by the tensile force Z into the somewhat recessed brake pan or trough 48, undulated in mirror image, with its centre point M₀ and the radius R₀, entering there into engagement.

In order for the self-locking eccentric means to function even in the absence of the tensile force Z, a further external tensile or compressive force may also act on the brake disk 37 or the eccentric means 43, e.g. according to FIG. 11 a any desired additional spring F, compressing or pulling the brake disk 37 into the brake pan 48. This results in slowing down or blocking (e.g. viewed from the heel side) any involuntary twisting in the absence of the main tensile force Z.

According to FIG. 10c a one-sided (right or left-hand) convexity and, as a result, also a lateral twisting may be set by means of the two other adjusting devices 35, designed in the present case as simple tensioning screws, in the longitudinal direction of the support or arching strip 39, e.g. into the position 39′ having the angle a as a “left-hand” torsion, which may be set and/or fixed at will. Alternatively, a “right-hand” torsion and/or arching of the arching strip 39 may be set with the other tensioning screw of the adjusting device 35.

If both tensioning screws of the adjusting devices 35 are slacked off sufficiently, i.e. if they cause no one-sided, forced torsion and the arching adjustment is only performed by the eccentric means 43, the arched support strip 39 may for each set degree of arching in addition twist passively in lateral direction, e.g. while walking. Vice versa, in the zero-position of the eccentric means 43 a one-sided and also two-sided arching may be brought about by tightening one or even both tensioning screws.

This results in that e.g. even an originally completely symmetrical shoe insert may be arched or twisted in itself in the same way or differently and even asymmetrically both for the left and the right foot.

The eccentric means 43 is adjustable by a reversible or stationary, radially arranged hand lever 46 and/or a multi-side spanner, which must engage in the centre of the eccentric means 43 in a matching spanner recess 47 (FIG. 10c). The latter is to be provided, in particular, if the shoe insert is permanently integrated in the shoe.

FIGS. 11a, b, c show an embodiment of a self-locking brake disk actuation mechanism comprising an eccentric means such as it may be fitted, for example, in a shoe insert according to FIGS. 10a, b, c, d. In a base strip 36 three partly overlapping bores (holes) are present with three different centre points (M₀, M₁ and M₂). In FIG. 11 a M₀ is simultaneously the centre point e.g. of a circular brake disk 37 including an eccentric bolt (which may also be a cable drum), in which case the circumference of the brake disk 37 has a cylindrical or conical braking surface 37′, e.g. having a sinusoidal or other wave-like, tooth-, or key groove-shape. In the direction of a tractional or tensioning means Z, approximately parallel to the axis AA the brake disk 37 having the radius R₀ is arranged in a recess (brake trough) 48 and is, as a result, self-locking or self-blocking against rotation. The brake trough 48 has a slowing down length BL, ranging from Kₗᵢ to Kᵣₑ. The other two bores with somewhat higher, offset and laterally spaced apart centre points M₁ and M₂ and the radii R₁ and R₂, which are approximately equally great or somewhat greater than the radius R₀ of the brake disk 37, on both sides form one aperture each, somewhat raised and laterally recessed in relation to the centre point M₀ with a smooth, cylindrical or conical edge, which, e.g. by milling, blanking or by means of an injection-moulding tool, is moulded into the base strip 36 of the shoe insert or in another brake casing or brake body. In the direction Z the pan has a slightly recessed, from Kₗᵢ to Kᵣₑ mirror-image like, sinusoidal or undulated conical or cylindrical edge surface. By way of the tensile force Z in the direction of the axis AA or approximately parallel to that axis, e.g. by tension exerted by a Bowden cable or the shoe insert tongue 40′ (FIG. 10), for example on the eccentric means 43, a pin or a cable or other traction or pressure element emerging from a cable drum, a toothed wheel with a rack or any other setting mechanism, manufactured as an integral part of the brake disk 37 or connected to the latter, the brake disk 37 is pushed into the pan in the direction of the tensile or compressive force Z, i.e. approximately parallel to the axis AA, so that it locks with its profile in the general region of the pan. As a result, the brake disk 37 cannot automatically rotate, regardless of how great the tensile force acting on the eccentric means 43 or on a cable drum whose diameter is preferably smaller than the length BL of the braking pan 48. Only torque attacking outside the brake disk diameter and independent of the tensile or compressive force Z and being greater than the latter, e.g. by means of the lever 46 or the hand wheel, may the brake disk 37 be lifted out of the pan beyond the tilting point Kₗᵢ to the left or Kᵣₑ to the right. The brake disk 37 is then lifted from the grid formation or brake lining or brake key groove in the pan towards the sliding surfaces 44 or 45 of the lateral bores with the centre points M₁ and M₂ and may now be turned as far as desired, since all marginal sine waves, key groove or brake lining points of the brake disk 37 slide through on the smooth inner circle 44 (left) or 45 (right) by way of their heads or otherwise configured outer edge. The same applies accordingly, if instead of the lever 46, e.g. a hand wheel, engages, whose diameter is greater than the diameter of the brake disk 37 or if a box spanner recess 47 (FIG. 10d) and a matching releasable box spanner is used for twisting the brake disk 37 instead of a reversible hand lever 46, 46′ or a hand wheel, not shown. Instead of a sinusoidal or otherwise configured structure a conventional brake material or brake lining may likewise be used. Other than in shoe inserts, a self-locking traction-, pressure- or adjusting device may be used wherever a self-locking displacement distance is desired or advantageous for any Bowden cable, toothed wheel, tooth rack, cable drum or any other adjusting devices under tensile or compressive load.

The particular advantage of all self-locking brake actuation means such as they are shown e.g. in FIGS. 10 and 11 (regardless of whether they comprise eccentric means-, cable drum-, tooth rack- or tensile or pressure transmission of whatever type) resides in that they operate in a self-locking manner continuously and at all points in both directions of rotation, also e.g. in the case of cable drums etc., even beyond greater displacement distances, e.g. beyond 360° and even beyond a plurality of revolutions. In addition, depending on the selection of the sinusoidal or otherwise configured symmetrical or asymmetrical configurations of the brake surfaces or of the material used, differently strong adjustment resisting means during twisting may be provided and/or different self-locking or even blocking properties, i.e. braking or blocking forces for diverse adjustments of the braking action.

The particular inventive step of this self-locking braking action resides in that braking, blocking and nevertheless easy twisting at any time beyond 360° of the brake disk works for as long as the tensile force Z attacks, i.e. pulls or presses in its tractional direction Z inside the brake trough between Kₗᵢ and Kᵣₑ, which according to the construction shown (FIGS. 11a, 11b and 11c) is ensured at all times.

FIG. 12 shows an embodiment of a double-arch and twisting support, consisting of two bending elements 54, 54′ arranged in each case rigidly or slidably on parallel axes of a structure 53, adapted to be interconnected in the embodiment. FIG. 12 a by a lower, joint rib 55. The rib 55 may likewise be arranged at any level or may be dispensed with altogether. The side wings 8, 8′ of the bending elements 54, 54′ are of approximately uniform, in relation to one another symmetrical, shape and are so arranged that they are able to adapt passively to a determined body shape pressed against them from outside or they may be forced actively into a determined given shape, which may be controlled by one or a plurality of pressure or traction elements 13, 13′, 14, 14′ individually or in any desired combination. The latter may be arranged to lock in any desired manner or they may be adjustable incrementally or continuously with or without self-locking action. By way of two Bowden cable arrangements 56, 56′, guided in the present case towards a joint adjusting button 57, the arching of both bending elements 54, 54′ may be adjusted jointly. Instead of the embodiment shown in this FIG. 12a comprising two support elements, embodiments including a plurality of support elements, e.g. three, five or more, are likewise conceivable, whose axes may not be arranged in a plane, but may also be at angles to another.

FIG. 12b shows a side elevation of the double-support according to FIG. 12a, wherein the possible angles of adjustment of the bending elements 54, 54a are indicated. Each bending element 54 or 54a (FIGS. 12b and c) may also be arched individually, e.g. by a separate Bowden cable (cables 52, 52′) or traction and pressure elements or any desired separate adjusting and tensioning devices.

As is apparent from the circle in FIG. 12b (enlarged in FIG. 12e), the wings 8, 8′ may be movably arranged, e.g. clipped on, individually or grouped together to turn or pivot about a wire, rod or tube and may be deformed by separate devices. Between the individual wings 8, 8′ filler elements 8a may be fitted on the bending elements 54, 54′, 54a etc., transferring the counter-pressure during bending e.g. via the bending (twisting) traction element 52.

Preferably the side wings 8, 8′ consist of a resilient material, such as e.g. a synthetic material or metal and may be designed in one piece as shown in FIG. 12a or may be composed of a plurality of individual components as indicated in FIG. 12b. The pressure or traction elements 13, 13′, 14, 14′, not shown, but indicated by arrows, required for changing the shape of the side wings 8, 8′ may be rigid, extensible, optionally lockable in set positions, e.g. by counternuts or limiting stops made of metal, synthetic material, glass fibre-reinforced materials, e.g. also in the form of a cable 52, according to FIG. 12b of a ribbon, chain or the like, in which context torsion springs, rigid shafts, screw-, hydraulic-, pneumatic- electric or other elements may be used.

The adjustment may be performed in a non-variable or variable, i.e. adjustable manner by changing e.g. press button connections or also by hooking into the fixing points 15 or by hooking bicycle spoke heads into bayonet-type slits or by traction and/or pressure elements 13, 13′, 14, 14′ arranged in any desired manner. Any desired motors, preferably electric motors or hand driven mechanisms are conceivable as drive means, in which context even accessories such as cogwheels and tooth racks, spools and operating cables, Bowden cables, levers, lever rod assemblies, eccentric devices, hydraulic means, pneumatic means, electric motors or electromagnets or the like may be used. Both damping pads and adjustable or self-locking locking mechanisms may be provided. In order to attain more elasticity, any desired, likewise adjustable and/or programmable spring elements may be provided, yielding to short-term pressure or traction, inside or outside the traction or pressure chain or traction or pressure elements or the arching-, twisting-, deforming mechanisms and their individual components.

FIGS. 13 and 14 show further embodiments of strips 1, in which case the strip 1 shown in FIG. 13 comprises in its lower region a plurality of partly off-centre and deflectable holding elements 3 with apertures 4 or brackets or bayonet-type holes or hooks, not shown, permitting additional distortions, rotations or torsions (see for example the arrows in FIGS. 1, 8, 9 and 13).

The embodiment illustrated in FIG. 14 shows a transverse arching or lumbar or support strip 32 which may be arched, twisted or otherwise deformed, wherein an adjusting device is indicated by means of which e.g. the arching or twisting, rotation or upright deformation may be adjusted by a traction element, e.g. a cable 33, “deflected” by a deflecting arch 34 from the axis A into, for example, an axis B. In the present case e.g. a simple tensioning screw 29 as shown in FIG. 9a or a double-threaded (right and left-hand threads) tension bolt as shown in FIG. 6 is provided to serve as the tensioning element, in which case the said tension bolt permits double advancement per revolution and nevertheless a complete self-locking action like a normal screw with single thread pitch.

FIG. 15a shows the shoe sole 61 of a shoe, in which case the shaft or the upper leather has been omitted. An insole 62 with a longitudinal slit 63 in front and a recess 64 at the back has been integrated in the shoe sole 61. On the insole 62 an arching strip 65 is arranged, which in the present embodiment is fixed in the heel region by a nipple 66 to prevent longitudinal displacement. The front end of the arching strip 65 is arranged by a traction spoke 67 on the front section of the insole 62 in a longitudinally displaceable, mobile manner. In this context, the one end of the traction spoke 67 is passed through the front longitudinal slit 63 of the insole 62. In the embodiment the traction spoke 67 is connected to the arching strip 65 by means of a spoke head 68 which is designed as an integral part of the traction spoke 67 or separately from the latter. The spoke head 68 may be anchored in the arching strip by a bayonet-type locking means. The other end of the traction spoke 67 is connected to a tensioning device 69. In the embodiment, arranged underneath the insole 62 in the shoe sole 61, it takes the form of a double-threaded tensioning device 69. The latter is in co-active engagement with the rear recess 64 in the insole 62. In the longitudinal axis of the double-threaded tensioning device 69, out towards the heel end, a left-hand thread hole is provided through the nipple 66 and then further a through-hole in the heel region of the shoe sole 61 for the head of a left external-thread screw 71 comprising a right internal-thread for the right external traction bolt 70 and e.g. a hexagonal bolt, which is prevented from rotating in its hexagonal sliding passage. Due to the right-hand turning of the double-threaded tensioning device 69 straight tensioning of the traction spoke 67 is now performed with double right-plus-left-thread pitch and with its hooked-in spoke head 68 a displacement of the arching strip 65 on the insole 62 towards the heel, resulting in an intensification of the arching of the arching strip 65. When turned (to the left) in the opposite direction, reduced arching is brought about by its displacement on the insole 62 towards its front end.

The head of the left-threaded tensioning screw 71 arranged rotatably in the shoe sole 61, may be adjusted e.g. by means of a box spanner or a slit, optionally also a cross-slit, e.g. by a screwdriver or a coin. By a marking or a scale around the exit aperture of this screw 71 at the end of the shoe, repeatable adjustment is possible. A double-threaded tensioning device 69 is provided, because in comparison with a likewise usable screw with a single thread it permits with the same number of revolutions twice the tensioning distance and, as a result, greater arching or twice the degree of de-arching during release per revolution.

In the embodiment shown in FIGS. 16a and b likewise an insole 62, conventional in shoes, is arranged in the shoe sole 61. Here and in what follows all components which are repetitive in all embodiments, have the same reference numerals. On the insole 62 there is likewise arranged an arching strip 65, designed in a displaceable manner at its front end of the insole 62. Underneath the insole 62, at an angle a in relation to the longitudinal axis of the arching strip 65 a tongue 72 is arranged, permanently connected by its front end to the front region of the arching strip 65. The rear end of the tongue 72 comprises a recess 73, suited and intended to be connected to a downwardly projecting pin 74 of an arrangement of an eccentric means 75, e.g. to be suspended from it, in which context any other manner, releasable or permanent, is conceivable as well. The arrangement of the eccentric means 75 is arranged in a rotatable manner within the end region of the arching strip 65. In the region of the arrangement of the eccentric means 75 and, in the present case, its permanently connected pin 74, the rear recess 64 is situated in the insole 62. By way of an exemplary arrangement of the tongue 72 with its recess 73 on the pin 74, the arching of the arching strip 65, by adjusting the arrangement of the eccentric means 75, may be adjusted in both directions for intensification, or, respectively, reduction of the arching, in which case at the same time, due to the angular arrangement of the tongue 72 in relation to the central axis a spatial distortion of the arching strip 65 may be performed. The arrangement of the eccentric means 75 may include a brake disk arrangement, preventing involuntary adjustment.

Because of the lateral incisions 77, at least in the end region of the insole 62, the arching strip 65 is rendered flexible, which promotes twisting. The eccentric means 75 may be twisted e.g. by a multi-side spanner, which may e.g. be introduced from the top. The incisions 77 may also serve to additionally perform spherical or other spatial deformations, e.g. by depressions or elevations performed in the desired regions.

The embodiment shown in FIGS. 17 and 18 includes once again an insole 62 and also an arching strip 65, at the front end of which a tongue 72 passed through the front longitudinal slit 63 is connected to the arching strip 65 either as an integral part thereof (see FIG. 18) or e.g. by riveting 76 (see FIG. 17), in which context both the front and the rear end (heel end) of the arching strip 65 are supported on the insole 62 in a mobile manner. In FIG. 17 at the rear end of the heel region the arching strip 65 (in contrast to the arrangement in FIG. 18) is rigidly connected to a rear tongue 72′ (without internal thread) by riveting 76; in-between a spacer is provided, making it possible for the insole 62 to move freely in this region. In FIG. 17 both tongue ends are adjustably arranged on different threads of a one-piece double-threaded screw 70, in which case the insole 62 does not participate in an adjustment of the arching.

In the embodiment according to FIG. 17 the ends of the front and rear tongue 72 and 72′ adjustably arranged on the thread parts are brought closer together or, respectively, moved away from one another by turning the double-threaded screw 70, from which results that a more pronounced or a reduced arching of the arching strip 65 may be set.

In the embodiment according to FIG. 18 the one end of the rear tongue 72′ is supported on the screw head 80 of the screw 78, ensuring the free turnability of the latter, which, preferably possesses a thread only in the front region, permitting by way of the front tongue 72 an adjustment of the arching by turning the screw 78.

In the embodiment according to FIG. 18 only the single-threaded screw 78 is rotated, tensioning or, respectively, releasing thereby the front tongue 72, causing the front section of the arching strip 65 on the insole 62 to get displaced and therefore arched or de-arched, while the rear end of the rear tongue 72′ supports the screw head 80. By the insertion of springs or resilient discs, the entire arching support becomes resilient.

The adjusting elements and their kinematic connections are arranged in a cavity underneath the insole 62 in the heel region of the shoe sole 61.

In the embodiment according to FIG. 19 a and b the insole 62 includes merely a rear recess 64. In this case the arching strip 65 is guided into the front section (toe section) of the insole 62 and is fixed or fastened there, while it is supported in the rear region on the insole 62 in a mobile and adjustable manner. In the heel region the arching strip 65 comprises a rear tongue 72′ connected to the latter in a permanent or releasable manner. At the free end, passed into the cavity in the heel region, the rear tongue 72′ is provided with a tapped bush 82. Into the bush 82 a headless pin 83 is screwed, prevented from longitudinal displacement by a traction clasp 81, but shifting, e.g. from the heel region, when twisting the traction tongue 72′ to the front towards the tip of the toes by its tapped bush 82 and displacing the arching strip 65, to which the tongue 72′ is rigidly connected in one or more pieces, likewise towards the tip of the toes on the stationary insole 62, causing the latter to arch and, vice versa, making it possible to set a lesser degree of arching when turning it back.

In this embodiment the arching strip 65 comprises passage holes, for example for better ventilation, as can be seen, in particular, in FIG. 19b.

In the embodiment shown in FIGS. 20a and b, in addition, even a multiple, spatial distortion is possible. In the shoe sole 61 once again an insole 62 is provided including a front longitudinal slit 63 and a rear recess 64. In this case, under the insole 62 behind the longitudinal slit 63 at least one cavity, guided into the heel region, is provided, serving to accommodate tensioning or adjusting devices. This cavity and the tensioning or adjusting devices may also be arranged at angles to one another, making it possible to set during tensioning, apart from arching, also a differently pronounced distortion and also an upright deformation of the arching strip 65. On the insole 62 an arching strip 65 is arranged, whose front portion is permanently connected to a front tongue 72, arranged at an angle in relation to the longitudinal axis. At the other free end of the front tongue 72 a holding device 84 is arranged in a rotatable manner, serving to rotatably accommodate, e.g. by a thread connection, the one end of a traction screw 85. At the rear end of the arching strip 65 passing through the rear recess 64 a rear tongue 72′, in the present case likewise permanently connected to the arching strip 65, is guided into the lower heel cavity, through which the other, i.e. the head end of the traction screw 85 is placed. In this case the arching strip 65 forms an integral part of the front and rear tongue 72, 72′. An access in the heel region to the head of the traction screw 85 makes it possible to so twist the latter that the arching of the arching strip may be intensified or, respectively, reduced. This allows multiple deflection of forces, i.e. multiple distortion, in which context, in order to perform further, e.g. stationary, local distortions, the arching strip 65 may comprise diverse incisions or recesses. Because of the heel a cavity permits the access to the traction screw 85, making it possible to attain an arching of the arching strip 65 and simultaneously a rotation.

FIG. 20a shows an arching strip 65, shortened to approximately ⅔ and FIG. 20b an arching strip 65 in shoe length.

The embodiment shown in FIG. 21 provides an insole 62 and an arching strip 65. In the embodiment the arching strip 65 comprises a front tongue 72 in the front region forming an integral part of the arching strip 65. In the rear region the arching strip 65 is permanently connected to a rear tongue 72′, arranged underneath the insole 62, but adapted to move freely on and in the insole 62, the free front end of the rear tongue 72′ being guided through a further recess in the insole 62 towards the arching strip 65 and the front tongue 72 through e.g. the same recess in the insole 62 into a cavity in the shoe sole 61. E.g. by way of a screw 78 having a very coarse pitch and a recoil brake the ends of the front tongue 72 and the rear tongue 72′ are adjustably interconnected. Passing through a central aperture in the arching strip 65 the screw 78 and, consequently, the arching of the interior of the shoe may be adjusted, for example by means of a screwdriver 86.

The embodiment shown in FIG. 22 comprises in schematic plan view an operating cable 79, guided via a guide means 87 to two locations in the heel region, with the tensioning of which, e.g. by reeling at one end, the arching of the arching strip 65 and with slowing down or blocking of the operating cable in the deflection or guide means 87 a left-hand or right-hand twisting may be set and adjusted entirely at will, since the guide means 87 is connected to the arching strip 65. In the path region of the operating cable 79 different regions may be raised or lowered as well.

If the traction or pressure elements (spokes 67, tongues 72, 72′, operating cables 79) enter into engagement with the arching strip 65 in a rotatable and an articulated manner, a medically important, “passive” twisting-in-itself (back and forth movement) of the foot-sole in the arching region with each step and depending on the load may be presented as inventive novelty, despite the degree of arching set in each case by twisting the front or rear arching strip section about the articulated, otherwise rigid traction or pressure element connection, in spite of an optionally set and so remaining respective arching.

If these points of engagement of the tongues 72, 72′ or spokes 67 or operating cables 79 are slowed down or blocked in their deflection 87, both this twisting and back and forth movement of the foot about the longitudinal axis in the arching region may be slowed down or prevented or, conversely, be actively varied in a predetermined manner.

The operating cable 79 may also consist separately of two operating cables, which, each on its own, may be adapted to be tensioned or tension-released, in which context by way of the cable deflections which may be slowed down or blocked and by a combination of individual possibilities a more systematic and multiple twisting may be actively and passively controlled as well.

Further embodiments, not shown, may comprise one or a plurality of traction or pressure elements alone or in combination, by means of which optionally a plurality of single torsions, combined also with simultaneous arching or torsion(s) may take place. For that purpose not only the described traction or pressure elements may be used, but any rigid, flexible or resilient traction, pressure or twisting element serving those purposes.

This invention makes it possible to continue to manufacture all shoes with “conventional” insoles or with special one-piece or multi-piece insoles, arching and twisting element combinations in assembly line mass production, as was the case to date, and without any appreciable extra time and cost expenditure and to be adjusted in an anatomically optimal way by the respective purchaser or owner of each shoe according to his own comfort or upon medical advice individually by himself or only with a special box spanner by the orthopaedist or prosthetist.

This novel, variable, individually adjustable or back and forth arching-, twisting-, deforming-possibility is of particular value to shoe soles and inserts in that, e.g. by gradual, e.g. monthly or otherwise systematic adjustment or resetting, similar to orthodontic braces etc. a malformation of healthy feet may be prevented already from early childhood and continuously thereafter or that existing deformations may be gradually reversed again by medical doctors and prosthetists as slowly as they have developed.

According to university professors even an improvement of public health in this sector might be possible.

The different possibilities of adjusting the arching from the shoe interior or from outside, even under load or while standing upright on such an arching-twisting sole and the possibility of a precise setting by an orthopaedist or medical doctor in front of a fluorescent screen until the “correct” arrangement of the individual bones or foot elements has been reached, presents a novelty as well in comparison with the inserts or shoes deformed irreversibly by plaster of Paris- or electronic-, computer, 3D-procesess etc. Conversely, the novel possibility of adjusting the arching or twisting of the arching or support elements from inside the shoe (see e.g. FIGS. 3, 4, 11 and 12) has its advantages as well.

FIGS. 23a and b show an arching-twisting support for a foot arching on the underside of an integrated cover sole 88 with a perforated short arching/twisting strip 65 and self-locking eccentric wheel-traction-adjustment of the arching.

Setting and adjusting the arching, distortion or upright deformation of this “short” support, in particular of the arching strip 65, may in this case be performed by rotating the eccentric brake wheel 75, namely both from the upper side through an aperture in the cover sole 88 and in the arching strip 65 as well as from the underside, e.g. if used as a loose shoe insert, e.g. by means of a hexagonal or other engagement box spanner or a coin in the adjustment slit.

This eccentric brake wheel 75 has a self-locking action when both traction tongues 72 and 72′ are pulled away in longitudinal direction, because the resulting direction of traction from the eccentric means at all times pulls the outer, larger sinusoidal or toothed or brake-lining-coated cylindrical friction surface into a central trough of the second traction tongue into the brake or friction surface pan, thereby locking the eccentric wheel against rotation.

However, by means of a box spanner or a relatively large coin the eccentric brake wheel 75 together with the eccentric means may now be tipped, via the lever arm, which is now larger than the brake trough, out of its brake wheel trough and locking position in both directions of rotation during twisting into the smooth supporting circle, which has no braking properties, thus permitting twisting and eccentric means adjustment at will. After the adjusting lever has been released, the brake wheel, due to the central tractional force of the two traction tongues, is immediately, automatically returned to the brake trough and locked.

In addition, FIG. 23a shows an arching-twisting support, in principle “symmetrical”, suitable both for the left as well as the right foot, as this support or arching strip 65 in the event of twisting the eccentric wheel to the right clock-wise in relation to a right-hand shoe insert for the right foot may be arched and twisted if a box spanner is put through the aperture 89 in FIG. 23b from the upper side, i.e. from the cover sole 88 and is then twisted. For twisting the eccentric wheel to the left anti-clockwise by means of the same box spanner, the symmetrical arching-twisting strip is converted into a left-hand shoe insert for the left foot.

Twisting in relation to a right-hand shoe sole is brought about in that up to an approximately 180° right-hand twisting with the above box spanner the eccentric means describes a semi circle viewed on the inner side of the right foot in the direction of walking, i.e. viewed from below in FIG. 23a in the longitudinal upper half above the axis AB, causing the two traction tongues 72 and 72′ in the direction of walking and viewed from above to arch in a more pronounced manner eccentrically on the inner side of the right foot than is the case on the outside. Vice versa, in the event of an approximately 180° left-hand twisting of the eccentric wheel the symmetrical arching strip is twisted at any degree of arching into a left-hand arching-twisting support for the left foot.

FIG. 23b shows the same extremely short arching-twisting support according to FIG. 23a in a side elevation and sectionalised along the longitudinal-central axis AB, but formed in one piece or by means of a spigot 91 and 91′ or a Velcro or similar detachable connection element releasably or permanently connected to a resilient cover sole 88, e.g. made of cork, felt, leather, synthetic material, wool, fabric, cardboard or the like, in which context this loose, very flat shoe insert comprises in the present example also a through-hole 89 from the top through the cover sole 88 in the event that this loose flexible and stretchable cover sole or cover is to be fitted permanently, together with the arching-twisting support, in a shoe or elsewhere in the technical or medical field.

FIG. 23b further shows the hooks 90 or 90′, which are releasable in the present example, but may also be formed in one piece, into which the traction tongues 72 and 72′ are hooked and which may also be connected to the arching strip 65 by way of riveting, gluing or welding.

FIG. 24a shows a section through an arching-twisting support, which already possesses a maximally pre-arched flat leaf spring serving as the arching strip 92, which may be manufactured from steel, hard aluminium, hard plastics etc. and which is de-arched by tensioning the de-arching screw 98 via the traction tongue 94 and the traction hook 93. This generates a novel, two-fold comfort, firstly in that the de-arching is still possible by means of the adjusting screw, similar to the arching of arching strips to date, and secondly, because further resilient de-arching is possible due to the free space 96 in the traction tongue 94 with tension release-tolerance if the spring arching strip 92 is loaded beyond its inherent resilient force, until the traction hook 93 comes to rest on a limiting pin 95.

FIG. 24b shows this two-stage super comfort on the same de-arching-twisting-deforming support in a weighted state. A weight X, simulating the pressure of a foot during walking or standing, shows the elastic yielding of the de-arching strip 92 from the optionally adjustable arching illustrated by a broken line up to a fully drawn out, flatter degree of arching, i.e. up to the limiting stop of the resilient traction hook 93 fully exploiting the free space 96 according to arrow Y up to the limiting stop 95.

FIG. 25a shows a support with a pre-arched spring-loaded arching strip 92, which at its arched ends may be spread apart, i.e. de-arched and arched again at will by rotating the eccentric wheel 75 via two pressure tongues 101 and 101′, e.g. by means of a box spanner 102. In addition, this support may passively under load be even further de-arched in each arch setting as a result of an at least one-sided free space “+” inside the sliding hook 104.

FIG. 25b shows the same spring-loaded arching strip viewed from below with the insole 96 having been omitted.

FIG. 26 shows an arching-twisting strip 65, archable at will by the tensioning device 69 and the traction tongues 72 and 72′, in its fully arched state, whose apex “S 0” may shift automatically in the longitudinal/walking direction (i.e. passively) up to “S 1” towards the front or “S 2” towards the back in the direction of the heel in that the free arching curve of the arching strip 65, maintaining the apex or level of arching over the arching length may shift its location and may deform by itself, automatically synchronously with the foot position, foot movement and form of the load. If loaded according to the white arrow 1 in FIG. 26 the arching apex shifts up to “S 1” and if loaded in the direction of the white arrow 2 up to “S 2”.

FIG. 27 shows an arching-twisting support, wherein an optional apex-displacement between “S 1” and “S 2” and likewise of the length of this distance “a” between S 1 and S 2 is possible for each level of arching set by the tensioning device 69 due to two longitudinally displaceable sliding braces 105 and 105′ pressing the arching strip 65 down at these locations in that by a further longitudinal adjustment device (not shown) both the length of the connection element 106 as well as the two sliding braces 105, 105′ connected to the latter are displaced at the same time and that the arching, now actively controlled, may be displaced within the range of the respective adjusting possibility of the control element, e.g. by an amount “a”.

FIG. 28 shows a longitudinal section through an arching-twisting support, whose arching strip 65 may, on the one hand, be reset to any desired basic degree of arching by means of a screwdriver passing through the latter itself and rotation of the traction screw 69 and possesses, due to an interposed buffer spring 117, in the event of an external load an additional resilient de-arching possibility beyond the set basic degree of arching in the scope of the structurally provided tension-release free space, denoted by the double arrow + at the front end of the foot, where the arching strip may slide on the insole 62, while being permanently connected to the insole 62 in the heel region. This permits, due to the weight exerted by a foot, resilient, additional super comfort for each step with the respectively set arching.

The extraneous drive (mini-tooth rack 110, mini-cogwheel 111, shaft 112, worm 113 and worm wheels 114) integrated in this embodiment (see FIGS. 28a and b) is rotated further by way of power generation with each resilient comfort step and with the up- and down movement of the arching strip 65 and the mini-tooth rack 110 connected to it as well as the cogwheel 111 with return-stop via a flexible shaft 112, a worm screw 113, a worm wheel 114 by one unit per step, displacing in the process a sliding strip 108 on or under the arching strip 65 in longitudinal direction per revolution via the eccentric pin 115 of the worm wheel 114. In these sliding strips 108 integrated knobs or projections or rotatable rollers releasably connected to the former, guided in their turn through slits in the arching strip 65, may perform massaging movements on the cover sole or on the underside of a foot during displacement within the slits.

FIG. 28a shows the section AA viewed from the heel and FIG. 28b the section BB through the mini-tooth rack 110 and through the cogwheel 111.

In FIG. 29 a novel possibility of arching is illustrated, wherein the arching-twisting-deforming strip is divided into two overlapping arching strips 92 and 92′, which may be displaced relative to one another by tensioning a pressure screw 126 (e.g. by a screwdriver) and lengthened in the process in their overall length and their arching enlarged in that both their ends are retained. In the heel region it is permanently connected to the insole and in the front foot region it is permanently connected to a stationary traction tongue 128.

Due to the connection of this traction tongue to the integrated extraneous elements, bellows 118, pneumatic-cylinder 120, pistons including piston rod 121, pressure pipes 119, relay valve 127 including switch and chip 125 and 125′, which are in co-active engagement with one another, a predetermined, additional arching and de-arching of any already individually preset arching of an arching strip 65 or 92 or the divided arching strip 92 and 92′ is attained when compressing the resilient and compressible shoe sole and the bellows 118 by a pumping action over a pre-programmed number of walking steps or by reaching a determined air pressure and/or sensor limit, and attaining additional arching or de-arching depending on the set chip-programme or sensor, after the mini-air pressure drive mechanism has been switched on incrementally until the relay valve 127 switches over within the desired limits. Instead, separate sliding strips 108 or 108′ with knobs or rolls may likewise be displaced under or on the arching strips by the mini-air pressure drive mechanism.

FIG. 30a shows a further embodiment in longitudinal section and FIG. 30b in plan view, comprising an integrated extraneous element, e.g. a mini-electric or compressed air motor 122 with internal power supply 123, e.g. a battery, an accumulator, a compressed air cartridge etc., and a connection socket 124 for external power supply, e.g. a power unit, battery charger, compressed air etc. coupled to an external switch 125 or to an internal switch 125′, which may be actuated in the shoe by means of a box spanner or a coin, by means of which both the power supply as well as a programmed chip may be switched on and set, e.g. for rhythmic, alternating, vibrating, increasing or decreasing, intermittent or even power supply, and as a result twisting of the worm 113, rotating one or two worm wheels in a self-locking manner and making it possible in the process to displace approximately parallel via their eccentric pin and a connecting tongue the two sliding strips 108 and 108′ against one another below the arching strip.

Any pressure and/or massaging knobs 109 or rotatably mounted rolls 109′ arranged on these sliding strips protrude through slits into the arching strip 92 and transmit their compressive force during the displacement in opposite direction below the arching sole for each set level of arching and, depending on the setting of the motor, the eccentric stroke, the chip-programme, their massage movements to the sole of the foot or a cover sole integrated thereabove.

In the embodiment shown in FIGS. 30a, b, regardless of the controlled movements by extraneous drive mechanisms, the de-arching strip 92 shown here, possessing a pronounced inherent arching, may be expanded in its basic arching by a sinusoidal eccentric wheel 75 as well as—in the present case—by two pressure tongues 101 and 101′ by means of a box spanner 102 in both directions of the arrows and may therefore be adjusted to a desired basic arching, e.g. as illustrated.

In this case as well, the inherent arching of the spring-loaded arching strip 92 in the event of a greater load than the inherent arching force due to its further displaceability in the front foot region, may be even further resiliently flattened as super comfort in the region of the available free space in both directions according to the double arrow denoted by the + symbol. Once the overload has been cancelled, only the basic arching set in each case is always restored. The latter can only be adjusted by a box spanner. However, in spite of all set or resilient archings of the arching strip, as a result of the mini-drive mechanism and the internal or external energy as well as the switch programming by chip-control connected through slits in the arching strip or directly to the arching strip, the movements performed additionally by the extraneous element may be carried out at will.

A self-locking drive mechanism of this or a similar type may, instead of the sliding strips, arch or de-arch, distort, deform in upright position or vibrate the entire de-arching strip 92 or, in different designs, the arching strip 65 entirely as programmed.

The plan view shown in FIG. 30b onto a fitted, complete arching-twisting support (in a shoe) with shortened front foot portion and open heel region, shows stepwise from top to bottom first the spring-loaded arching strip 92, in order to illustrate the longitudinal sliding strips 108, 108′, then also the insole 62, which is cut, in order to also show the mini-electric motor 122, the worm screw 113 and the worm wheels 114 together with their traction-pressure tongues and the housing 130 for the motor including worm and worm wheel.

1. Support—in particular for medical or technical applications—comprising at least one flexible element which can be arched and which comprises at least one central part and lateral elements, the arch of said element being adjustable by means of a device, characterised in that even in the arched state at least one lateral element and/or central element may be at least partially spatially distorted, and/or twisted and/or deformed about an axis.

2. Support according to claim 1,
characterised in that an additional further element is integrated.

3. Support according to claim 1,
characterised in that the spatial distortion, twisting or deformation may be performed freely between limiting stop formations.

4. Support according to claim 1 or 3,
characterised in that the spatial distortion and/or twisting and/or deformation about an axis may be set as a whole and/or in individual regions by at least one longitudinally adjustable and/or an angularly adjustable mechanism.

5. Support according to any one of claims 1, 3 or 4,
characterised in that individual or all lateral elements consist of separate side wings, with or without interposed filler elements, and are arranged on a separate bending element.

6. Support according to any one of claims 1, 3 to 5,
characterised in that it comprises arrangement points for tensioning and/or adjusting means.

7. Support according to any one of claims 1, 3 to 6,
characterised in that the tensioning and/or adjusting means are fixed in a releasable manner.

8. Support according to any one of claims 1, 3 to 7,
characterised in that, at least one tensioning and/or adjusting device comprises double-threaded screws.

9. Support according to any one of claims 1, 3 to 8,
characterised in that the support further comprises a self-locking eccentric drive mechanism.

10. Support according to any one of claims 1, 3, to 8,
characterised in that the support further comprises a self-locking cable drum- and/or tooth rack drive mechanism.

11. Support according to claim 2,
characterised in that the additional further element is an insole of a shoe.

12. Support according to claim 11,
characterised in that the insole simultaneously forms part of the support.

13. Support according to claim 11 or 12,
characterised in that the support comprises apertures for passing there through traction, pressure, distortion, support or tensioning elements in the insole, the latter being slidably connected to the support.

14. Support according to claim 11 or 12,
characterised in that the support comprises apertures for passing there through traction, pressure, distortion, support or tensioning elements in the insole, the latter being immovably connected to the support.

15. Support according to claim 13 or 14,
characterised in that holding elements for the traction, pressure, distortion, support or tensioning elements are arranged in the apertures.

16. Support according to claim 11 o 15,
characterised in that the support consists of two sections, one section being arranged below the insole and one section above the insole.

17. Support according to any one of claims 2, 11 to 16,
characterised in that the lateral and/or central and/or additional further element is/are adjustable in the shoe from the outside.

18. Support according to claim 17,
characterised in that the adjustment may be performed by a tool in the form of a box spanner or a coin.

19. Support according to claim 17 or 18,
characterised in that the degree of adjustment may be read off a marking device.

20. Support according to any one of claims 2, 11 to 19,
characterised in that the spring elements possess adjustable elasticity.

21. Support according to any one of claims 2, 11 to 20,
characterised in that springs and/or resilient elements are positioned between operative elements.

22. Support according to any one of claims 2, 11 to 17,
characterised in that at least a portion of the outer and/or inner edge of an element comprises incisions and projections increasing the elasticity.

23. Support according to claim 1,
characterised in that additional extraneous elements are integrated or present.

24. Support according to claim 23,
characterised in that an insole or cover sole serves as an extraneous element.

25. Support according to claim 24,
characterised in that the insole or cover sole forms simultaneously part of the support.

26. Support according to claim 24 and 25,
characterised in that the support comprises apertures for passing there through traction, pressure, distortion, support or tensioning elements in the insole, the latter being partially slidably connected to the support.

27. Support according to claim 25 or 26,
characterised in that holding elements for the traction, pressure, distortion, support, tensioning, control and/or drive elements are arranged in the apertures.

28. Support according to any one of claims 24 to 27,
characterised in that the support consists of two sections, one section being arranged below the insole and the other above the insole.

29. Support according to any one of claims 24 to 28,
characterised in that the lateral and/or central and/or additional extraneous element is adjustable in the shoe from the outside or from the inside.

30. Support according to claim 29,
characterised in that the adjustment of support-, extraneous- or drive- or control elements may be performed by means of a box spanner, coin, electric, mechanical or pneumatic external or internal switch and/or remote control.

31. Support according to claim 29 or 30,
characterised in that at least one location of the support or extraneous element is moved periodically or alternatingly directly or via interposed elements such as a worm/worm wheel, an eccentric wheel, a cogwheel/tooth rack, a cable/cable disc, pneumatic/hydraulic means etc. in co-active engagement with a motor or drive mechanism and an internal or external power or other energy source.

32. Support according to any one of claims 24 to 31,
characterised in that at least one adjustment of the support or extraneous element is performed directly or indirectly by chip-, sensor- or other programmed control.

33. Support according to any one of claims 24 to 32,
characterised in that a free space is provided between the operative elements permitting additional, resilient, passive comfort de-arching for each set degree of arching of the support.

34. Support according to any one of claims 24 to 33,
characterised in that individual support or extraneous elements are themselves spring elements.

35. Support according to any one of claims 24 to 34,
characterised in that the arching element consists of an already pre-arched steel or other spring element and may be de-arched by the tensioning or adjusting elements or devices.

36. Support according to any one of claims 24 to 35,
characterised in that the arching element consisting of two pre-arched, overlapping arching strip parts may be lengthened or shortened in its overall length and arched by a pressure screw.

37. Support according to any one of claims 24 to 36,
characterised in that an arching strip and/or sliding strips, each with massaging elements, may, in addition, be moved automatically with each walking step by an integrated air- or mechanical drive mechanism at each set degree of arching according to preset programming.

38. Support according to any one of claims 24 to 36,
characterised in that due to the integrated motor drive mechanism with adjusting programming and/or sensors and internal or external power supply the arching strip or another element of the support may additionally be moved at will in each position of arching.

39. Support according to any one of claims 24 to 38,
characterised in that all elements and integrated extraneous elements of the support designs are designed according to a modular construction system and may be interchanged or combined at will.
